(19) United States
(12) Patent Application Publication
(10) Pub. No.: US 2011/0190652 A1
(43) Pub. Date: Aug. 4, 2011

(54) SYSTEM AND METHOD FOR ACQUIRING AND DISPLAYING UTERINE EMG SIGNALS

(75) Inventors: Rainer J. Fink, College Station, TX (US); Jack N. McCrary, Houston, TX (US); Jay Porter, College Station, TX (US); Mark Burns, McKinney, TX (US)

(73) Assignee: REPRODUCTIVE RESEARCH TECHNOLOGIES, LLP, Houston, TX (US)

(21) Appl. No.: 12/696,936

(22) Filed: Jan. 29, 2010

(51) Int. Cl. A61B 5/0488; A61B 5/053

(52) U.S. Cl. 600/546; 600/547

(57) ABSTRACT

A system and method for acquiring and processing uterine EMG signals from a maternal patient. Raw uterine EMG signals are acquired and processed in a central unit designed to isolate the patient and any internal circuitry from electrical shock. The central unit has a circuit board that amplifies and filters the EMG signal, then transmits the signal to an AID converter, after which the digitized signal is transmitted to a computer for further processing of the signal and subsequent display of a signal representative of uterine activity. The system and method provide a more accurate measurement of uterine EMG signals than a tocodynamometer or IUPC, and are useful in predicting delivery or monitoring the patient during post partum uterine activity.

### BACKGROUND

During late pregnancy and the labor process, there are generally two methods of acquiring and monitoring uterine activity. The first method involves the use of a tocodynamometer (hereinafter referred to as a “toco”). The toco is a non-invasive device fastened to the abdomen of pregnant patient by means of an elastic strap and used to measure uterine contraction frequency. The typical toco consists of an external, strain-gauge instrument, or a pressure transducer, designed to measure the stretch of the mother's stomach and indicate when a uterine contraction has occurred. When the skin stretches, the pressure transducer records an electrical signal whose waveform can be evaluated by the treating physician.

The toco, however, has many drawbacks. One disadvantage is that it is an indirect method of pressure reading and is therefore subject to many interfering influences which can falsify the measuring result. Its effectiveness can be entirely dependent on the tightness of the belt used to place the toco on the maternal abdomen. Also, the effectiveness of the toco is dependent on transducer location and, therefore, does not function once the baby has descended down the uterus and into the birth canal where no pressure transducer is present to report pressure variations. Moreover, the toco is highly inaccurate and fails to function properly on obese patients since the pressure transducer requires that uterine contractions be transmitted through whatever intervening tissues there may be to the surface of the abdomen.

The second method involves the use of an intrauterine pressure catheter (hereinafter referred to as an “IUPC”). A typical IUPC consists of a thin, flexible tube with a small, tip-end pressure transducer that is physically inserted into the uterus next to the baby. The IUPC is configured to measure the actual pressure within the uterus and thereby indicate the frequency and intensity of uterine contractions. However, in order to place the IUPC, the amniotic membrane must be ruptured so that the catheter can be inserted. Improper placement of the IUPC catheter can result in false readings, thereby requiring repositioning. Similarly, the catheter opening can become plugged and provide false information requiring the removal, cleaning and reinsertion of the IUPC, Lastly, inserting the catheter runs the risk of severely injuring the head of the baby, and also carries with it a significant infection risk. Thus, generally the IUPC is rarely used, and can only be used at delivery.

What is needed, therefore, is a system that overcomes the above-noted disadvantages of the toco and IUPC. In particular, a system is needed that overcomes the inaccuracy of the toco, especially in instances with obese patients, and further overcomes the invasive and precarious nature of the IUPC.

### SUMMARY

Embodiments of the disclosure may provide a system for acquiring and processing uterine EMG signals from a patient. The system may include a pair of electrodes in communication with a skin impedance matching system, wherein the pair of electrodes are configured to acquire a raw EMG signal from the patient, a signal processing module communicably coupled to the pair of electrodes and configured to filter and amplify the raw EMG signal to obtain a processed EMG signal, and to convert the raw EMG signal, or a processed EMG signal, from an analog signal to a digital signal, and a computer communicably coupled to the signal processing module and having software for executing machine-readable instructions to receive, process, and subsequently display the processed EMG signal.

Embodiments of the disclosure may further provide a method of acquiring and processing uterine EMG signals from a patient. The method may include applying at least one pair of electrodes to a maternal abdomen of a patient, matching the skin impedance of the patient, obtaining a raw analog uterine EMG signal, processing the raw uterine EMG signal in a signal processing module to obtain a digital EMG signal, transmitting the digital EMG signal to a computer having software for executing machine-readable instructions, and processing the digital EMG signal in the computer to obtain a signal representative of uterine activity.

Embodiments of the disclosure may further provide another system for acquiring and processing uterine EMG signals from a patient. The other system may include a signal processing module having an internal processing circuit, an EMG communication port coupled to the signal processing module and operatively coupled to the processing circuit, at least one pair of electrodes communicably coupled to the EMG communication port and configured to acquire and transmit a raw EMG signal from the patient to the processing circuit, where the processing circuit amplifies and filters the raw EMG signal to a frequency band between about 0.2 Hz to about 2.0 Hz to obtain a processed EMG signal, an analog to digital converter operatively coupled to the processing circuit and configured to convert the processed signal into a digital EMG signal, and a computer communicably coupled to the signal processing module and having software for executing machine-readable instructions to receive the digital EMG signal from the analog to digital converter and further process the digital EMG signal by filtering and amplifying to a frequency band between about 0.3 Hz to about 1.0 Hz to obtain a signal representative of uterine activity.

### BRIEF DESCRIPTION OF THE DRAWINGS

The disclosure is best understood from the following detailed description when read with the accompanying Figures. It is emphasized that, in accordance with the standard practice in the industry, various features are not drawn to scale. In fact, the dimensions of the various features may be arbitrarily increased or reduced for clarity of discussion.

FIG. 1 illustrates a schematic of the uterine electrical activity analyzer system according to one or more embodiments of the disclosure.

FIG. 2 illustrates a schematic of the circuit board illustrated in FIG. 1.

FIG. 3 illustrates a schematic diagram of a portion of the power distribution module disclosed in FIG. 2.

FIG. 4 illustrates a schematic diagram of a portion of the power distribution module disclosed in FIG. 2.

FIG. 5 illustrates a schematic diagram of a portion of the power distribution module disclosed in FIG. 2.

FIG. 6 illustrates a schematic diagram of a portion of the power distribution module disclosed in FIG. 2.

FIG. 7 illustrates a block circuit diagram of a portion of an embodiment of the circuit board disclosed in FIG. 2.

FIG. 8 illustrates a block circuit diagram of a portion of an embodiment of the circuit board disclosed in FIG. 2.

FIG. 9 illustrates an exemplary schematic electrical circuit for a skin impedance matching system, according to at least one embodiment of the present disclosure.

FIG. 10 illustrates an exemplary electrical schematic of a resistor ladder network, according to at least one embodiment of the present disclosure.

### DETAILED DESCRIPTION

It is to be understood that the following disclosure describes several exemplary embodiments for implementing different features, structures, or functions of the invention. Exemplary embodiments of components, arrangements, and configurations are described below to simplify the disclosure, however, these exemplary embodiments are provided merely as examples and are not intended to limit the scope of the invention. Additionally, the disclosure may repeat reference numerals and/or letters in the various exemplary embodiments and across the Figures provided herein. This repetition is for the purpose of simplicity and clarity and does not in itself dictate a relationship between the various exemplary embodiments and/or configurations discussed in the various Figures. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact. Finally, the exemplary embodiments presented below may be combined in any combination of ways, i.e., any element from one exemplary embodiment may be used in any other exemplary embodiment, without departing from the scope of the disclosure.

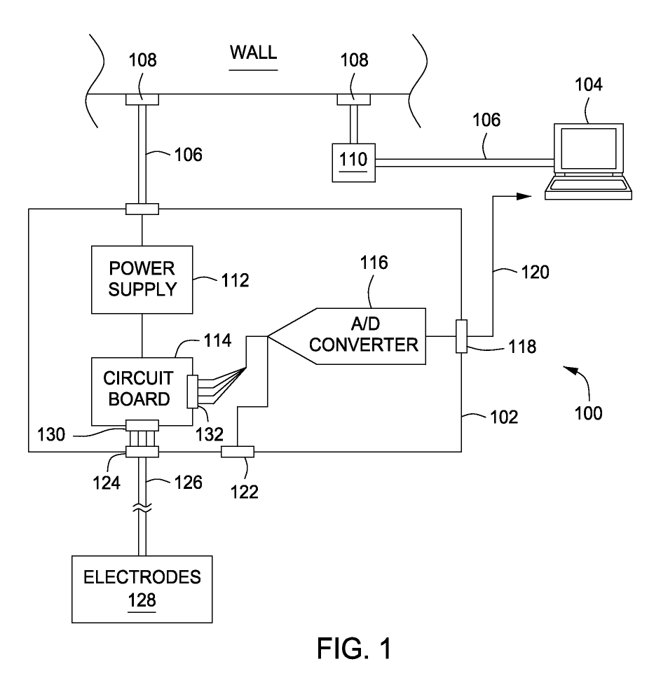

Referring to FIG. 1, illustrated is a system 100 for acquiring and processing uterine electromyography or electromyogram (“EMG”) signals. As known by those skilled in the art, EMG can also be known as or substantially similar to electrohistography or electrohistogram (“EHG”). Consequently, the acquisition and processing of EHG signals is also contemplated by the Inventors, without departing from the scope of the present disclosure. A uterine EMG signal is the functional equivalent to a uterine activity signal created by a toco or IUPC, but can be a great deal more precise. As explanation, uterine contractions comprise coordinated contractions by individual myometrial cells of the uterus. These global muscle contractions are triggered by an action potential and can be seen externally as an EMG signal. When electrodes are placed on the maternal abdomen, they measure the global muscle firing of a uterine contraction, thereby resulting in a “raw” uterine EMG signal.

The system 100 may include a signal processing module 102 communicably coupled to a computer 104. The signal processing module 102 and the computer 104 may each include hardware; however, the computer 104 may include software for executing machine-readable instructions to produce a desired result. In at least one embodiment, the software may include an executable software program created in commercially-available LABVIEW®. The hardware may include at least processor-capable platforms, such as client-machines (also known as personal computers or servers) and hand-held processing devices (such as smart phones, personal digital assistants (PDAs), or personal computing devices (PCDs), for example). Further, hardware may include any physical device that is capable of storing machine-readable instructions, such as memory or other data storage devices. Other forms of hardware include hardware sub-systems, including transfer devices such as modems, modem cards, ports, and port cards. In short, the computer 104 may include any other micro processing device, as is known in the art. The computer 104 may include a monitor for displaying processed uterine EMG signals for evaluation.

In an exemplary embodiment, the computer 104 may include, without limitation, a desktop computer, laptop computer, or a mobile computing device. Moreover, the computer 104 may include a CPU and memory (not shown), and may also include an operating system (“OS”) that controls the operation of the computer 104. The OS may be a MICROSOFT® Windows OS, but in other embodiments, the OS may be any kind of operating system, including without limitation any version of the LINUX® OS, any version of the UNIX® OS, or any other conventional OS as is known in the art.

Both the signal processing module 102 and the computer 104 may be powered via a medical-grade power cord 106 that may be connected to any typical wall outlet 108 conveying 120 volts of power. As can be appreciated, the system 100 may also be configured to operate on varying voltage systems present in foreign countries. For the computer 104, however, the power cord 106 may include an interim, medical-grade power brick 110 configured to reduce or eliminate leakage current originating at the wall outlet 108 that may potentially dissipate through the internal circuitry of the system 100 or a patient.

The signal processing module 102 may house a power supply module 112, a circuit board module 114, and an analog to digital (“A/D”) converter 116. The power supply module 112 may be configured to supply power for the signal processing module 102. In particular, the power supply module 112 may receive 120V-60 Hz power from the wall outlet 108 and convert that into a 12 volt direct current to be supplied to the circuit board module 114. In alternative embodiments, the power supply module 112 may be configured to receive varying types of power, for example, DC current from a battery or power available in foreign countries. As will be described in more detail below, the circuit board 114 may be any type of electronic circuit and configured to receive, amplify, and filter the incoming uterine signals.

The AID converter 116 may digitize the incoming analog uterine signals into a viewable digital signal transmittable to the computer 104 for display. Specifically, the AID converter 116 may be communicably coupled to an external USB port 118 located on the body of the signal processing module 102. In an exemplary embodiment, the USB port 118 may connect to a commercially-available USB 6008 (DAQ), available through NATIONAL INSTRUMENTS®. A double-ended USB connection cable 120 may be utilized to communicably couple the USB port 118 to the computer 104. As can be appreciated, however, the disclosure also contemplates alternative embodiments where the USB port 118 may be replaced with a wireless adapter and signal transmitter to wirelessly transmit the processed uterine data directly to a receiver located on the computer 104.

The signal processing module 102 may also include a toco communication port 122 through which physicians may be able to acquire and process uterine signals via a tocodynamometer (“toco”) or IUPC, as is already well-known in the art. For example, through the toco communication port 122, physicians may be able to track maternal and fetal heart rates, and also acquire intrauterine pressures via an IUPC or chronicle uterine activity via a toco. The analog signals sent to the toco communication port 122 may be directed to the AID converter 116 to be digitized and subsequently displayed through the computer 104. As described above, the digitized signals may be routed to the computer 104 via the USB port 118 and double-ended USB connection cable 120.

Similarly, and more importantly for the purposes of the present disclosure, the signal processing module 102 may include an EMG communication port 124 which may be communicably coupled to at least one pair of electrodes 128 and a patient ground electrode via an EMG channel 126. Through the electrodes 128, physicians may acquire and process raw uterine EMG signals. Specifically, the electrodes 128 may be configured to measure the differential muscle potential across the area between the two electrodes 128 and reference that potential to patient ground. Once the muscle potential is acquired, the raw uterine EMG signal may then be routed to an input 130 for processing within the circuit board 114, as will be described below.

After processing within the circuit board 114, the processed uterine EMG signal may be directed out of the circuit board 114, through an output 132, and to the AID converter 116 where the analog uterine EMG signal may be subsequently digitized for display on the computer 104. The digitized uterine EMG signal may be transmitted to the computer 104 via the USB port 118 and double-ended USB connection cable 120, as described above. However, alternative embodiments contemplate transmitting the data wirelessly to the computer 104 via a wireless adapter and signal transmitter (not shown). In at least one embodiment, the processed uterine EMG signal may provide uterine contraction frequency and duration information.

Although only one EMG channel 126 is illustrated, the disclosure fully contemplates using multiple EMG channels 126 each EMG channel 126 being communicably coupled to a separate pair of electrodes 128. In an exemplary embodiment, there may be four or more separate EMG channels 126 entering the EMG communication port 124.

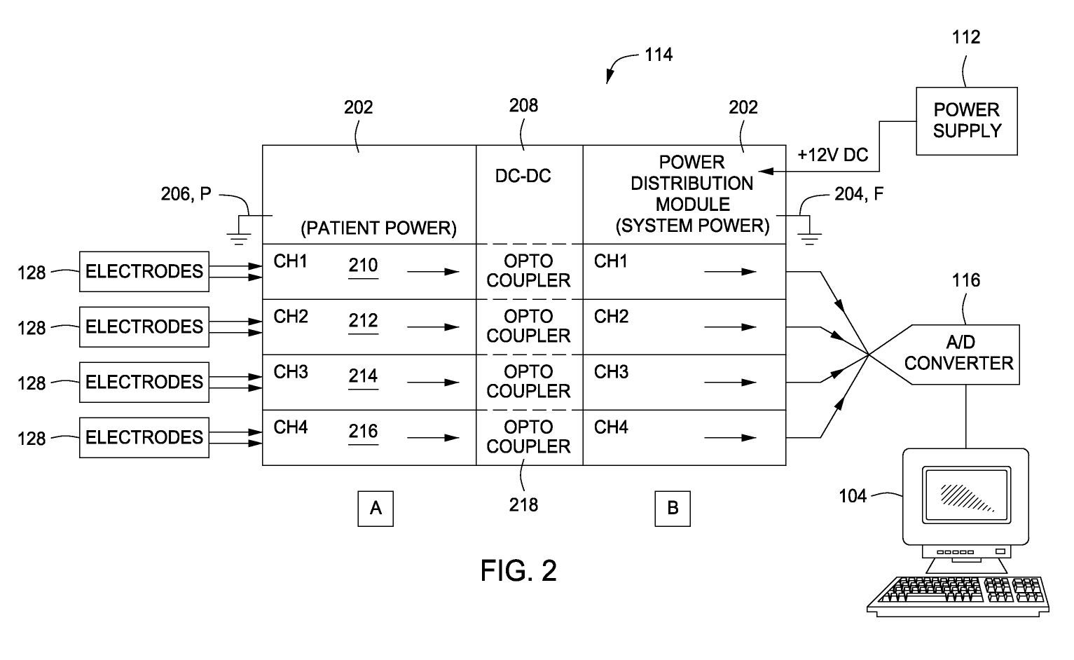

Referring now to FIG. 2, illustrated is an exemplary embodiment of the circuit board 114 located in the signal processing module 102, as described in FIG. 1. The circuit board 114 may include a patient side A, and a wall side B. As explained above, the circuit board 114 may receive a 12V direct current from the power supply module 112. In particular, the power supply module 112 may be communicably coupled to a power distribution module 202 located within the circuit board 114, wherein the power distribution module 202 may be configured to supply varying amounts of voltage to the internal circuitry of the circuit board 114. The power distribution module 202 may include a wall ground 204 and a patient ground 206, designed to not only protect the patient from stray leakage current but also to protect the internal circuitry from overload, as described below.

To help facilitate electrical shock protection for both the patient and the circuitry, the circuit board 114 may include an isolation DC-DC converter 208, or a transformer that separates the patient side A from the wall side B. In exemplary operation, the isolation DC-DC converter 208 may be configured to isolate power signals, thereby preventing stray charges from crossing over from one side and causing damage on the opposite side. In at least one embodiment, the isolation DC-DC converter 208 may include a commercially-available PWR1300 unregulated DC-DC converter.

As illustrated in FIG. 2, the circuit board 114 may be divided into a series of channels 210, 212, 214, 216. In the exemplary illustrated embodiment, four channels 210, 212, 214, 216 are indicated, labeled as CH1, CH2, CH3, and CH4, respectively, and may extend across both patient side A and wall side B. Each channel 210, 212, 214, 216 may be communicably coupled to a pair of electrodes 128, as described above. Once the “raw” uterine EMG signal is obtained by the electrodes 128, the differential signal is then delivered to each respective channel 210, 212, 214, 216 for processing and subsequent display.

Although four separate channels 210, 212, 214, 216 are herein disclosed, alternative embodiments may include more or less than four. In fact, suitable results may be achieved by employing a single-channel configuration. However, since inaccurate EMG signals can often result from poor skin impedance or misplacement of the electrodes 128, a plurality of channels 210, 212, 214, 216 may afford the physician with a plurality of opportunities to acquire an accurate uterine EMG signal. Furthermore, each channel 210, 212, 214, 216 may be separately-viewable on the computer 104 (FIG. 1) after signal processing has taken place.

Similar to the power distribution module 202, as a precautionary measure the channels 210, 212, 214, 216 on patient side A are isolated from their counterpart channels 210, 212, 214, 216 on wall side B by a linear optocoupler 218. In an exemplary embodiment, the linear optocoupler 218 may consist of a commercially-available IL300 optocoupler, available through VISHAY SEMICONDUCTORS®. As can be appreciated to those skilled in the relevant art, the linear optocoupler 218 may serve to avert potential electrical damage to the circuit 114 and the patient (not shown), as leakage current will be prohibited from transferring from one side A,B to the other B,A, or vice versa.

In exemplary operation, the linear optocoupler 218 may be configured to receive a partially processed EMG signal from the patient side A and create an optical light signal that transmits across the linear optocoupler 218 to the wall side B. To be able to optically transmit a signal across the linear optocoupler 218 from the patient side A to the wall side B, the incoming raw uterine EMG signal must first be amplified and filtered, as will be described in detail below. At the wall side B, the optical signal may then be converted back into an electrical signal and then undergo final amplification and filtration processes, as will also be described below. After final amplification and filtration on the wall side B, the processed uterine EMG signal may then be transmitted to the A/D converter 116 where the signal is digitized for display on the computer 104 (FIG. 1).

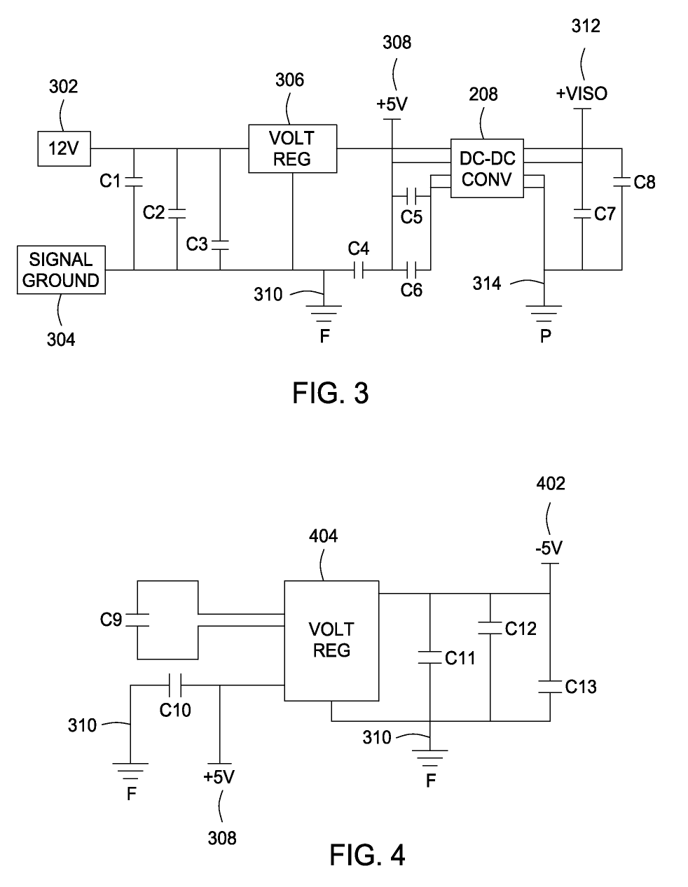

Referring now to FIGS. 3-6, illustrated are exemplary schematic diagrams for an embodiment of the power distribution module 202, as described above with reference to FIG. 2. To provide clean and safe power to the circuitry of the circuit board 114, the power distribution module 202 may be configured to filter and amplify the power signals several times. Clean power is desired so as to eliminate external noises introduced into the system via the power supply 112 (FIG. 1), thereby allowing the electrodes 128 to accurately register signals created only by the patient.

With reference to FIG. 3, the power distribution module 202 may include a 12V input power signal 302 and a signal input ground 304, both derived from the power supply 112 disclosed in FIG. 1. Although the 12V input power signal 302 was previously converted into a clean, medical-grade power via the power supply module 112, the power distribution module 202 may be designed to further clean the power so as to provide a safer source of power. To accomplish this, the 12V input power signal 302 may first be decoupled via a series of capacitors C1, C2, C3 arranged in parallel of decreasing capacitance, then be channeled through a voltage regulator 306 designed to reduce the 12V signal 302 to a +5V signal 308. As part of this process, the voltage regulator 306 may reference the +5V signal 308 to a partly-unsafe field ground 310.

Following the voltage regulator 306, a series of capacitors C4, C5, C6 may be connected and configured to further clean and filter the power, thereby creating a cleaner and more stable DC voltage. This leads to the isolation DC-DC converter 208, as described above with reference to FIG. 2. As previously explained with reference to FIG. 2, the isolation DC-DC converter 208 may be configured to isolate the 5V signal 302 on the wall side B, from the patient side A. The resulting clean and safe voltage is a +VISO signal 312, referenced to a patient ground 314, a safe grounding reference.

With reference to FIG. 4, the +5V signal 308 acquired in FIG. 3 may be converted into a −5V signal 402. The resulting signals 308, 402 may be used to power the circuitry located in the channels 210, 212, 214, 216 on the wall side B of the circuit board 114 (FIG. 2). In the illustrated embodiment, the +5V signal 308 is initially referenced to an unsafe field ground 310, but is subsequently filtered and amplified through a series of capacitors C9-C13 and a single voltage regulator 404. In an exemplary embodiment, the voltage regulator 404 may include a commercially-available LT1054 voltage regulator, available through TEXAS INSTRUMENTS®. The resulting −5V signal 402 may also be referenced to an unsafe field ground 310. The polar opposite signals may be required since amplifiers typically need dual-power supply signals to account for the positive and negative deflections to obtain the full sine wave. As will be seen below, the +5V signal 308 and the −5V signal 402 will be referenced by the several amplifiers located in the internal circuitry of each channel 210, 212, 214, 216 on the wall side B of the circuit board 114 (FIG. 2).

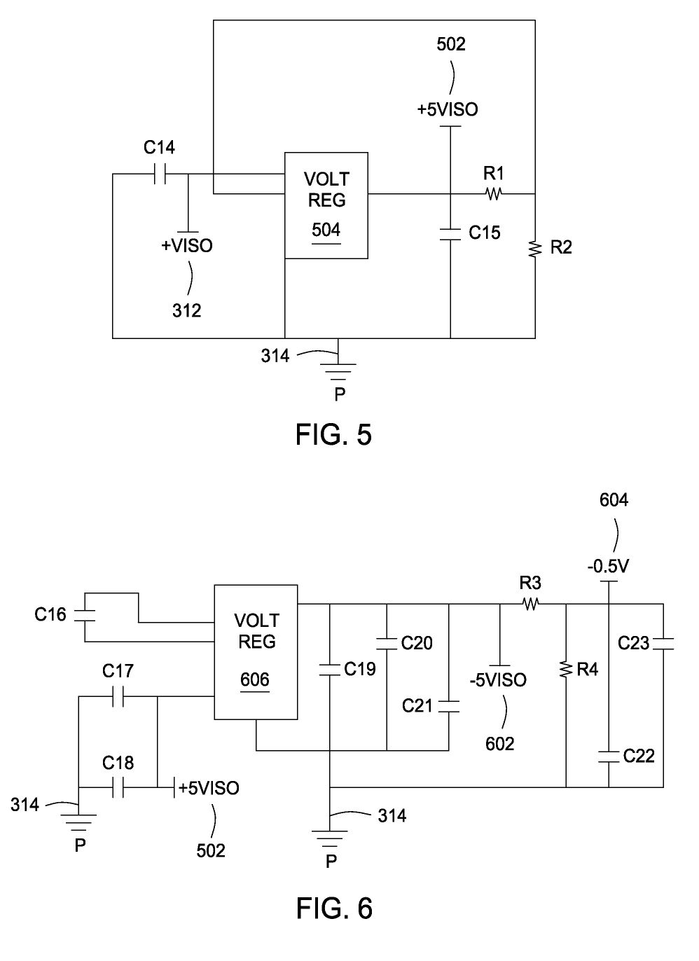

With reference to FIG. 5, the power distribution module 202 may be configured to use the clean +VISO 312 signal acquired in FIG. 3 and process it into a +5VISO 502 signal, a much cleaner signal including a very clean 5 volts of power. This may be accomplished, by filtering and amplifying the +VISO 312 signal through a series of capacitors C14, C15, a series of resistors R1, R2, and a voltage regulator 504. In at least one embodiment, the voltage regulator 504 may include the commercially-available LP2951 voltage regulator, available through NATIONAL SEMICONDUCTOR®. The resulting +5VISO 502 signal may be referenced to the very safe patient ground 314.

Lastly, with reference to FIG. 6, the power distribution module 202 may be configured to draw from the +5VISO 502 signal acquired in FIG. 5 above to create a −5VISO 602 signal and a −0.5V 604 signal. At the outset, the +5VISO 502 signal may be referenced to the safe patient ground 314. As illustrated in FIG. 6, the resulting signals 602, 604 may be created by filtering and amplifying the +5VISO 502 signal through a series of capacitors C16-C23, a series of resistors R3, R4, and a voltage regulator 606. In an exemplary embodiment, the voltage regulator 606 may include the commercially-available LT1054 voltage regulator, available through TEXAS INSTRUMENTS®. Moreover, the resulting −5VISO 602 signal and a −0.5V 604 signal may also both be referenced to the patient ground 314. As will be seen below, the +5VISO 502 signal and the −5VISO 602 signal will be referenced by the several amplifiers located in the internal circuitry of each channel 210, 212, 214, 216 on the patient side A of the circuit board 114 (FIG. 2). In at least one embodiment, the −0.5V signal may be acquired through a voltage circuit from the −5VISO voltage.

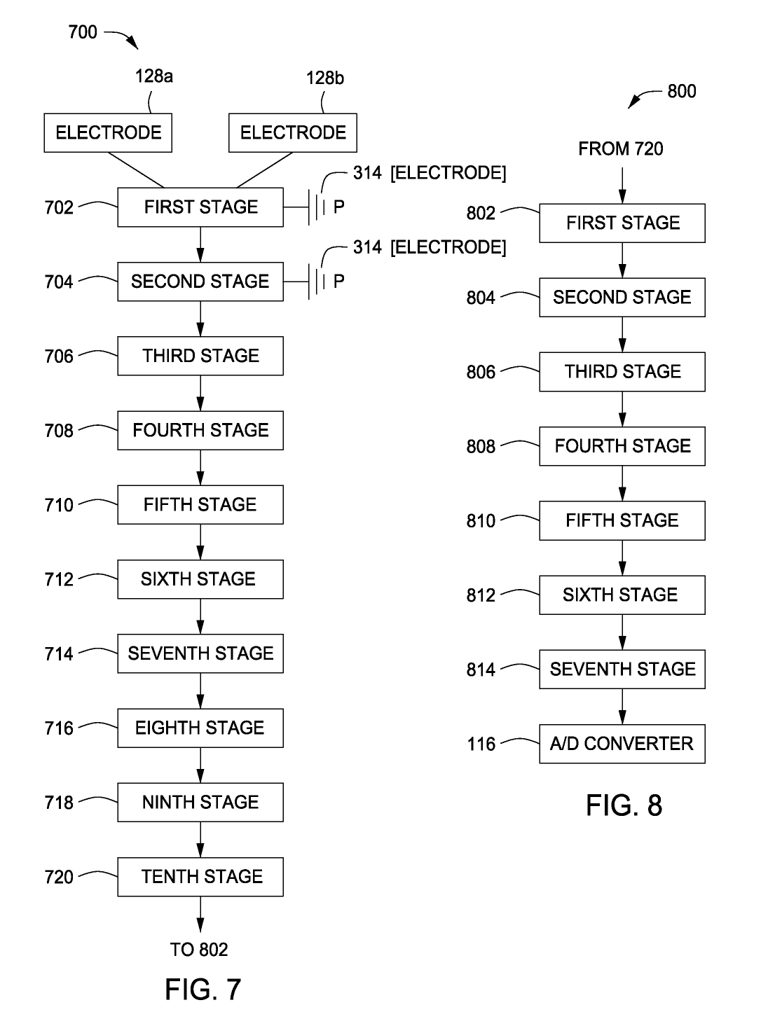

Referring now to FIG. 7, with continuing reference to FIG. 2, illustrated is a block diagram representative of the internal circuitry 700 located on the patient side A of the circuit board 114 for each channel 210, 212, 214, 216. As illustrated, the internal circuitry 700 may consist of several stages configured to receive and process a raw uterine EMG signal from a patient. As will be appreciated, however, although the internal circuitry 700 of only one channel 210, 212, 214, 216 is herein described, the description may nonetheless apply to each channel 210, 212, 214, 216.

As explained above, each channel 210, 212, 214, 216 may be communicably coupled to a pair of electrodes 128a, 128b that are designed to acquire the raw uterine EMG signals for processing. Specifically, the electrodes 128a,b may be configured to measure the differential muscle potential across the area between the two electrodes 128a,b and reference that potential to a ground electrode. As explained below, the electrodes 128a,b may also implement an impedance matching system that can provide relatively stable, impedance-independent output voltages to the internal circuitry 700. The first stage 702 may include an instrumentation amplifier configured to take the difference between the voltage seen at electrodes 128a,b and amplify the signal with reference to a patient ground 314, which may take the form of an electrode. To support the instrumentation amplifier in obtaining the differential amplification, the first stage 702 may include an arrangement of several capacitors and resistors.

Also included in the first stage 702 may be a series of diodes configured as a safety feature to ground out the circuitry in the event an unexpected voltage spike is introduced via the electrodes 128a, b. A typical diode voltage drop is 0.7V, allowing the diode act as a switch that opens when voltage is increased or decreased by at least 0.7V. For example, in the event of a positive voltage spike, such as an emergency defibrillation of the patient where approximately 1,000V may course through the patient's body and potentially enter the electrodes 128a,b, a positive diode may be configured to shunt any positive voltage above the typical 0.7V drop that enters via the electrodes 128a,b to ground. Upon encountering the positive diode, the power spike may be channeled away from the circuit board 114 (FIG. 1) and to the power supply 112 (FIG. 1) which is medically-isolated to the wall outlet 108 (FIG. 1), as described above.

However, as is known in the art, every time there is a voltage spike, the voltage will tend to peak and then return equally in the opposite direction until it stabilizes. To avoid sending an oppositely charged voltage spike back though the circuit board 114, or even to the patient through the electrode 128a,b, the circuitry in the first stage 702 may also include a negative diode configured to absorb any negative voltage spikes exceeding the 0.7V drop in the negative direction. In an exemplary embodiment, a set of positive and negative diodes may be provided for each electrode 128a,b.

Moreover, the first stage 702 may include at least one pull-up resistor dedicated to each electrode 128a,b, since in some cases the patient is incapable of creating enough energy to register a valid uterine EMG signal. Therefore, if needed, pull-up resistors may weakly “pull,” or draw out the uterine EMG signals from the patient.

The second stage 704 may be configured to provide further protection for the internal circuitry 700, and also further protect the patient from potentially dangerous leakage current traveling back through any electrodes 128a,b. In particular, the second stage 704 may include at least one resistor and a series of diodes, wherein the diodes may be designed to function similar to the diodes disclosed in the first stage 702 and further be referenced to a patient ground 314 designed to dissipate any stray peak voltages. Therefore, the second stage 704 may serve as a failsafe mechanism in the event the diodes in the first stage 702 fail to completely absorb any unexpected peak voltages.

The third stage 706 and the sixth stage 712 may each include a high-pass filter, while the fourth stage 708 and the seventh stage 714 may each include a low pass filter. Throughout the hardware defined herein, the combination of high-pass and low-pass filters may be configured to amplify and filter the incoming uterine EMG signals to frequencies broadly located between about 0.2 Hz to about 2 Hz, the typical frequency of uterine EMG activity found in humans. As can be appreciated, these filtration stages 706, 708, 712, 714 may eliminate some of the high or low frequency noises naturally emanating from the patient, or from the surrounding environment. In an alternative exemplary embodiment, the incoming uterine EMG signals may be amplified and filtered to frequencies located between about 0.2 Hz to about 2 Hz by means of a single band-pass filter, thereby replacing the various filtration stages 706, 708, 712, 714 with a single band-pass filter stage. In one or more embodiments, any variation or combination of the filtration/amplification stages 706, 708, 712, and 714 can be implemented to obtain the same or similar results, and still retain the same function. It will be appreciated that varying the stage order from that disclosed herein may, in at least one embodiment, result in enhanced outcomes,

The fifth stage 710 may include yet another voltage protection circuit, similar to the protection disclosed in stage three 706 above. In particular, the fifth stage 710 may provide a series of diodes and resistors configured to prevent the further influx of voltage surges, thereby protecting the internal circuitry 700 of the circuit board 114.

The eighth stage 716 may include a voltage divider configured to reduce the gain accumulated through the prior stages so as to provide the appropriate amount of voltage to the ninth stage 718. The ninth stage 718 may include a diode driver circuit having an operational amplifier (“op amp”) configured to adjust a diode configuration that is designed to feed data and power to an optocoupler located in the tenth stage 720. In exemplary operation, the op amp may not have enough capacity to power an optocoupler. The diode configuration in the ninth stage 718, therefore, may compensate for the lack in voltage stemming from the op amp and be powered by +5VISO 502 (FIG. 5) and referenced to −5VISO 602 (FIG. 6). Alternatively, the diode configuration in the ninth stage 718 may compensate for an excess of voltage stemming from the op amp, and dissipate excess voltage safely to ground so as to not damage the ensuing optocoupler.

The tenth stage 720 corresponds to the linear optocoupler 218, as explained above in FIG. 2. As described above, the optocoupler 218, also referred to as an optoisolator, may be configured to receive the partly-processed uterine EMG signal from the internal circuitry 700 located on the patient side A and create an optical light signal that transmits across the optocoupler 218 to the wall side B. It should be noted that no power is transferred over the linear optocoupler 218 from the patient side A to the wall side B. Instead, as explained above with reference to FIGS. 3 and 4, the wall side B is powered separately from the patient side A.

Referring now to FIG. 8, with continuing reference to FIG. 2, illustrated is a block diagram representative of the internal circuitry 800 located on the wall side B of the circuit board 114 for each channel 210, 212, 214, 216. As illustrated, the internal circuitry 800 may consist of several stages configured to receive the pre-processed uterine EMG signal from patient side A and process that data for analog to digital (A/D) conversion.

The first stage 802 and the fifth stage 810 of the internal circuitry 800 may include a low-pass filter designed to further filter the uterine EMG signal from any outlying noises, thereby focusing the signal frequency even closer to the broad frequency band lying between about 0.2 Hz-2.0 Hz. As will be described later, this frequency band may be filtered to a more narrow frequency band for more exact measurements.

The second stage 804 and the sixth stage 812 may include a buffer amplifier, respectively. As is known in the art, a buffer amplifier provides electrical impedance transformation from one circuit to another. Specifically, each buffer amplifier may be configured to prevent the preceding stages from unacceptably loading the ensuing stages and thereby interfering with its desired operation.

The third stage 806 and the fourth stage 808 of the internal circuitry 800 may be configured as calibrating stages designed to refine the incoming EMG signals. In particular, each stage 806, 808 may include a low-pass filter defined by at least one capacitor and at least one resistor. However, the third stage 806 may include a tunable DC offset, configured to be tuned by the use of a localized potentiometer. Also, the fourth stage 808 may include a tunable gain, wherein the amplitude of the incoming EMG signal may be altered so as to acquire a known amplitude. Thus, a trained technician or a doctor may be able to optimize the signal tuning at the hardware level. Although the frequency band may not be altered, the amplitude, gain, and DC offset may be manipulated to suit a particular application.

The seventh stage 814 may include a combination high-pass and low-pass filter configured to further filter the uterine EMG signal from any outlying noises, thereby focusing the frequency even closer to the broad frequency band lying between about 0.2 Hz-2.0 Hz.

The signal leaving the seventh stage 814 leads to the A/D converter 116 (FIG. 2) for digitizing. In an exemplary embodiment, the A/D converter may include a data acquisition (“DAQ”) card, such as the commercially-available NI 6008, available through NATIONAL INSTRUMENTS®, as described above. Following the A/D converter, as explained above, the processed signal may be transmitted to the computer 104 (FIG. 1) for software manipulation and display.

The computer 104 may be configured to initiate the LABVIEW® software program to acquire the digitized data and place it in an internal memory (not shown). The software may also be configured to algorithmically filter the incoming signal to between about 0.3 Hz and about 1.0 Hz to thereby obtain a more precise signal representative of uterine activity. During the filtration process, software manipulation of the data may include removing any motion artifacts, or stray signals resulting from patient movement or someone contacting the electrodes 128 or leads and thereby causing a spike in signal activity. To accomplish this, the software may be programmed with a uterine EMG threshold that automatically disregards registered signals that exceed that limit. Alternative software data manipulation may include altering the gain of the signal, and calculating the root mean square of the data to obtain a signal representative of uterine activity, as commonly seen in the toco and IUPC. Furthermore, it is also contemplated to acquire a signal substantially equivalent to the root mean square by taking a low-pass filter frequency (e.g., 0.01 Hz). Such an equivalent signal will also be similar to a signal as commonly seen in the toco and IUPC.

Thus, contemplated in the disclosure is hardware filtering and software filtering of the incoming EMG signals. Such multi-layer frequency filtering may have the advantageous effect of isolating only the signals representative of uterine activity. After full signal processing has taken place, the processed data in the form of a signal representative of uterine activity can be displayed, stored in memory for future reference, transmitted, or printed.

Regarding the electrodes 128 as described in FIGS. 1 and 2, they may further include a hardware-embedded software solution configured to continuously monitor the skin-to-electrode impedance. In monitoring the skin impedance, the electrodes 128 may be configured to alter the input impedance of the monitoring circuitry to dynamically adapt to the changing impedance mismatch between the patient and the electronics. In at least one embodiment, the skin-to-electrode impedance may be implemented in a continuous-monitoring mode or time-defined monitoring mode, to allow either real-time implementation of the impedance matching or predefined matching based upon the accuracy required by the medical procedure.

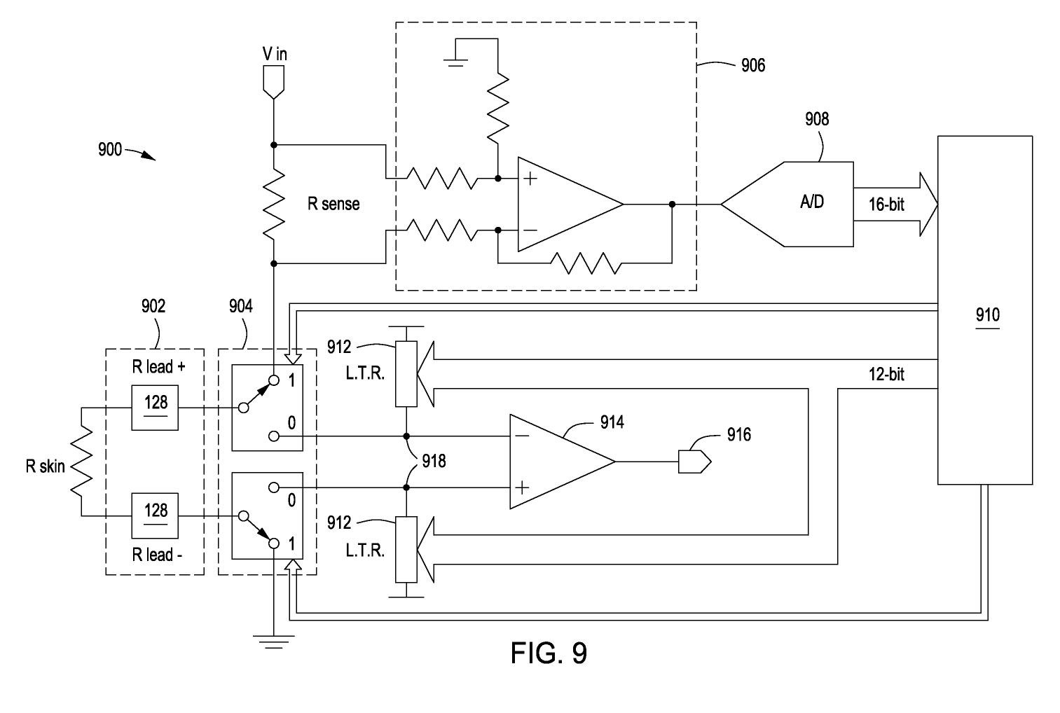

FIG. 9 illustrates an exemplary schematic electrical circuit for a skin impedance matching system 900. The system 900 may be configured to measure the skin-to-electrode impedance and adaptively alter the input impedance of the electrical monitoring circuitry to match the measured skin-to-electrode impedance. The system 900 may include a first matching module, or measurement circuit, having a skin-to-electrode interface 902 including electrodes 128, a pair of switches 904, a current sensing differential amplifier 906, an AID converter 908, and a microprocessor 910.

In exemplary operation, the measurement circuit senses the input impedance of the skin-to-electrode interface, amplifies, digitizes, and provides information to the microprocessor 910. Within the microprocessor 910, an embedded software routine may be configured to analyze the incoming data and generate a series of control signals to a communicably coupled resistor ladder network 912. In at least one embodiment, the control signals are a 12-bit communications.

Following the resistor ladder network 912 may be a second sensing module or mode, a differential amplifier 914 may be employed to amplify the incoming electrical signals generated by the patient. In an exemplary embodiment, a medical device 916, such as the signal processing module 102 (FIG. 1), may be attached to the amplifier 914 in order to obtain data from the electrodes 128. As can be appreciated, multiple circuits may be progressively switched using the same electrodes 128, if appropriate. In at least one embodiment, the amplifier 914 is not employed. In alternative embodiments, the amplifier 914 may be integrally-embodied in the medical device 916.

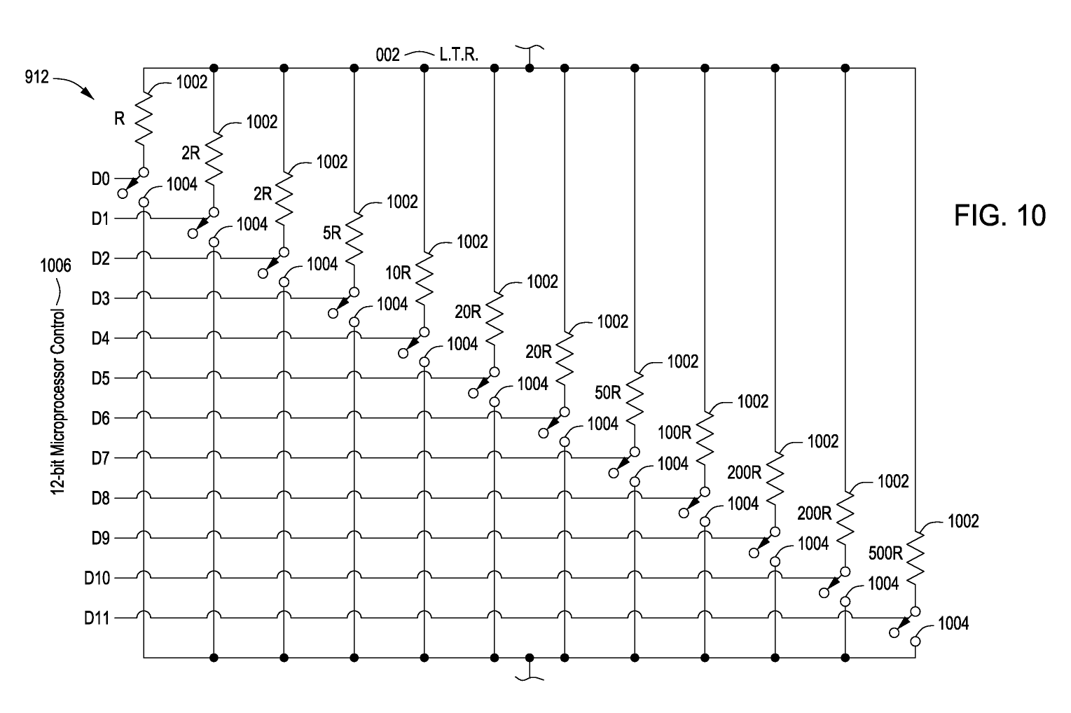

Referring now to FIG. 10, illustrated is an exemplary electrical schematic of at least one resistor ladder network 912. As shown, the resistor ladder network 912 may include a plurality of resistors 1002 and microcontroller-activated switches 1004 to implement a number of resistor 1002 combinations in parallel, thereby allowing tremendous accuracy in the total impedance generated by the combined resistor ladder 912. Depending on the application, the resistor 1002 values may vary. For example, in the illustrated exemplary embodiment, R may equal 1K Ohms, where the value of R may vary per application.

In exemplary operation, with continuing reference to FIGS. 9 and 10, the electrodes 128 communicably coupled to the electronic monitoring system 900 may first be placed on the patient skin surface. The microprocessor 910 may then be configured to adjust the switches 904 to the “ON” or 1 position, thereby creating a current flow path from Vin, through Rsense, Rlead+, Rskin, Rlead− to ground. In an exemplary embodiment, the microprocessor 910 may communicate to the switches 904 with 2-bit, or even 1-bit, signals. The voltage drop, and thus the current through the resistor Rsense, may then be measured and amplified by the current-sensing differential amplifier 906. The resulting analog signal may then be digitized by the AID converter 908 and passed in a multi-bit format to the microprocessor 910. An embedded-software routine in the microprocessor 910 may be configured to analyze the digitized information and thereby calculate the resistive load applied by the skin-to-electrode interface 902.

The microprocessor 910 may then create a set of control signals 1006 (FIG. 10) and send them to the resistor ladder network 912 in order to activate the switches 1004 as needed. Activating the switches 1004 may include creating a set of parallel resistors 1002 configured to generate an overall resistive load corresponding to the resistive load created by the skin-to-electrode interface 902. Upon completion of the resistive-matching operation, the microprocessor 910 may then set the input switches 904 back to the “OFF” or 0 position, thereby returning the electronic system 900 to regular operation as a medical monitoring device, while leaving the resistor ladder network 912 programmed to match the skin-to-electrode impedance.

The exemplary values of resistors 1002 disclosed in FIG. 2 may be configured to generate a variety of resistance values by various combinations of switches 1004 that are no more than a 5% variance with any skin impedance generally between 10K ohms to 100K ohms. Due to the matching operation, the voltage from monitoring the skin through the electrodes 128 may be split at the junctions 918 where a portion of the voltage flows through the network 912 and the other portion flows through the amplifier 914.

As explained above, the disclosure may work to satisfaction with a simple one-channel configuration having a pair of electrodes attached to the maternal abdomen. However, the inventors contemplate alternative applications including employing a plurality of channels, even more than the four channels 210, 212, 214, 216 disclosed herein. For example, in one embodiment a plurality of channels, through the electrodes 128 connected thereto, may be placed strategically amidst the span of the maternal abdomen for the purpose of monitoring the transmission speed of the uterine contraction as it moves longitudinally down the uterus. This may prove advantageous as it may allow a physician to pinpoint and localize where the uterus contraction begins and how that contraction moves along the length of the uterus. Since uterine contractions may push up or down, this may allow a physician to instruct a patient to push down when the uterus is also pushing down, thus avoiding counter-productive pushing by the mother and potential risk to the baby.

Moreover, it should be noted that it is contemplated by the inventors that the system 100 disclosed herein may be used during pregnancy and also post partum. Thus, the system 100 may be able to retrieve and display uterine activity after birth for physician reference.

The foregoing has outlined features of several embodiments so that those skilled in the art may better understand the detailed description that follows. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions and alterations herein without departing from the spirit and scope of the disclosure.

Page 23 of 29

We claim:

1. A system for acquiring and processing uterine EMG signals from a patient, comprising:
a pair of electrodes in communication with a patient abdomen, wherein the pair of electrodes are configured to acquire a raw EMG signal from the patient;
a signal processing module communicably coupled to the pair of electrodes and configured to filter and amplify the raw EMG signal to obtain a processed EMG signal, and to convert the raw EMG signal, or a processed EMG signal, from an analog signal to a digital signal; and
a computer communicably coupled to the signal processing module and having software for executing machine-readable instructions to receive, process, and subsequently display the processed EMG signal.

2. The system of claim 1, further comprising a skin impedance matching system comprising:
a matching module configured to determine the skin impedance by sensing an input impedance from the patient through the pair of electrodes, and amplifying and digitizing the input impedance;
a resistor ladder network configured to match the skin impedance using at least one resistor;
a microprocessor configured to analyze the input impedance and generate a series of control signals to direct the resistor ladder network to match the skin impedance; and
a sensing module configured to sense uterine EMG signals from the patient through the pair of electrodes in conjunction with the resistor ladder network.

3. The system of claim 2, wherein the sensing module is communicably coupled to the signal processing module.

4. The system of claim 1, wherein the signal processing module further comprises a toco communication port configured to provide an interface with a tocodynamometer or an IUPC to track uterine activity, wherein the tracking of the uterine activity may be displayed on the computer.

5. The system of claim 1, wherein the signal processing module further comprises a circuit board, power supply, and an analog to digital converter.

6. The system of claim 1, wherein the computer comprises a wireless receiver and the signal processing module further comprises a wireless transmitter for wirelessly transmitting the processed EMG signal to the wireless receiver of the computer.

7. The system of claim 1, wherein the signal processing module filters and amplifies the raw EMG signal to a frequency band between about 0.2 Hz to about 2.0 Hz.

8. The system of claim 6, wherein the circuit board comprises a patient side and a wall side, the patient side being separated from the wall side by an isolation DC-DC converter configured to isolate power signals and prevent stray charges from crossing over from the wall side to the patient side or from the patient side to the wall side.

9. The system of claim 8, wherein the circuit board further comprises a plurality of channels extending across the patient side to the wall side, wherein the plurality of channels are separated by an optocoupler configured to prevent potential electrical damage to the circuit board and the patient.

10. The system of claim 1, wherein the computer processes the processed EMG signal by filtering and amplifying the processed EMG signal to a frequency band between about 0.3 Hz and 1.0 Hz, and also by removing motion artifacts from the processed EMG signal.

11. The system of claim 1, wherein the software of the computer is programmed to determine the root mean square of the processed EMG signal to obtain a signal representative of uterine activity.

12. The system of claim 11, wherein the signal representative of uterine activity is displayed on a monitor communicably coupled to the computer.

13. A method of acquiring and processing uterine EMG signals from a patient, comprising:
applying at least one pair of electrodes to a maternal abdomen of a patient;
matching the skin impedance of the patient;
obtaining a raw analog uterine EMG signal;
processing the raw uterine EMG signal in a signal processing module to obtain a digital EMG signal;
transmitting the digital EMG signal to a computer having software for executing machine-readable instructions; and
processing the digital EMG signal in the computer to obtain a signal representative of uterine activity.

14. The method of claim 13, wherein the signal processing module is also configured to process input signals from a tocodynamometer or IUPC.

15. The method of claim 13, wherein matching the skin impedance of the patient comprises:
determining the skin impedance of the patient by sensing an input impedance from the patient, and amplifying and digitizing the input impedance;
analyzing the input impedance using a microprocessor;
generating a series of control signals to direct the resistor ladder network to match the skin impedance;
matching the skin impedance using at least one resistor in a resistor ladder network; and
sensing the uterine EMG signals from the patient through a sensing module coupled to the at least one pair of electrodes in conjunction with the resistor ladder network.

16. The method of claim 15, wherein the sensing module is communicably coupled to the signal processing module.

17. The method of claim 13, wherein processing the raw analog uterine EMG signal in a signal processing module comprises:
amplifying the raw analog EMG signal;
filtering the raw analog EMG signal to a frequency band between about 0.2 Hz to about 2.0 Hz to obtain an amplified and filtered analog signal; and
transmitting the amplified and filtered analog signal to an analog to digital conversion to convert the amplified and filtered analog signal into the digital EMG signal.

18. The method of claim 13, wherein processing the digital EMG signal in the computer further comprises filtering and amplifying the digital EMG signal to a frequency band between about 0.3 Hz and about 1.0 Hz.

19. The method of claim 18, wherein processing the digital EMG signal in the computer further comprises:
removing motion artifacts from the digital EMG signal; and
determining the root mean square of the digital EMG signal.

20. The method of claim 13, wherein the signal representative of uterine activity is displayed on a monitor communicably coupled to the computer.

21. The method of claim 13, wherein the signal processing module has a circuit board having a patient side and a wall side, the patient side being separated from the wall side by a isolation DC-DC converter configured to isolate power signals and prevent stray charges from crossing over from the wall side to the patient side or from the patient side to the wall side.

22. The method of claim 21, wherein the circuit board further comprises a plurality of channels extending across the patient side to the wall side, wherein the plurality of channels are separated by an optocoupler configured to prevent potential electrical damage to the circuit board and the patient.

23. A system for acquiring and processing uterine EMG signals from a patient, comprising:
a signal processing module having an internal processing circuit;
an EMG communication port coupled to the signal processing module and operatively coupled to the processing circuit;
at least one pair of electrodes communicably coupled to the EMG communication port and configured to acquire and transmit a raw EMG signal from the patient to the processing circuit, where the processing circuit amplifies and filters the raw EMG signal to a frequency band between about 0.2 Hz to about 2.0 Hz to obtain a processed EMG signal;
an analog to digital converter operatively coupled to the processing circuit and configured to convert the processed signal into a digital EMG signal; and
a computer communicably coupled to the signal processing module and having software for executing machine-readable instructions to receive the digital EMG signal from the analog to digital converter and further process the digital EMG signal by filtering and amplifying to a frequency band between about 0.3 Hz to about 1.0 Hz to obtain a signal representative of uterine activity.

24. The system of claim 23, wherein the computer is configured to display the signal representative of uterine activity on a monitor communicably coupled to the computer.
